(19) United States
(12) Patent Application Publication
(10) Pub. No.: US 2017/0035663 A1
(43) Pub. Date: Feb. 9, 2017

(54) METHODS OF TREATING GINGIVITIS USING HIGH SALT TOOTHPASTE

(71) Applicant: Colgate-Palmolive Company, New York, NY (US)

(72) Inventors: Yuyan Zeng, Guangzhou (CN); Chengkang Tan, Guangzhou (CN); Pingdong Li, Guangzhou (CN); Yun Xu, Guangzhou (CN)

(73) Assignee: Colgate-Palmolive Company, New York, NY (US)

(21) Appl. No.: 15/102,537

(22) PCT Filed: Dec. 11, 2013
(86) PCT No.: PCT/CN2013/089058

§ 371 Date: Jun. 8, 2016

(51) Int. Cl. A61K 8/19; A61K 8/34; A61K 8/86; A61K 8/25; A61K 8/60; A61K 8/46; A61K 8/73; A61Q 11/00; A61K 8/24

(52) U.S. Cl. CPC A61K 8/19; A61Q 11/00; A61K 8/345; A61K 8/86; A61K 8/24; A61K 8/60; A61K 8/463; A61K 8/731; A61K 8/25; A61K 2800/28; A61K 2800/48

(57) ABSTRACT

A method of treating gingivitis comprises applying a toothpaste comprising at least 5% sodium chloride in a toothpaste base comprising a non-silica calcium carbonate abrasive and humectant, to the oral surfaces, e.g., the teeth and gums, of a patient in need thereof, together with compositions for use therefor.

### FIELD

The present invention relates to methods of treating gingivitis using a toothpaste that contains high levels of sodium chloride in a calcium carbonate base.

### BACKGROUND

Gum disease affects a significant number of people worldwide, and is a leading cause of tooth loss. Gum disease usually begins with gingivitis, in winch bacteria in dental plaque buildup causing the gums to become inflamed. Dental plaque is a soft deposit winch forms on teeth and is comprised of an accumulation of bacteria and bacterial by-products. Plaque adheres tenaciously at the points of irregularity or discontinuity e.g., on rough calculus surfaces, at the gum line and the like. A wide variety of antibacterial agents have been suggested in the art to retard plaque formation. For example, halogenated hydroxydiphenyl ether compounds such as triclosan ate well known to the art for their antibacterial activity and have been used in oral compositions to counter plaque formation by bacterial accumulation in the oral cavity. Hoover, many people prefer to use natural products to provide antibacterial activity. Accordingly, there exists a need for methods of treating gingivitis using natural products having antibacterial activity.

### SUMMARY

Unless otherwise indicated, the terms “%” or “percent” when used in connection with an ingredient of the toothpastes for use in treating gingivitis is intended to refer to the percent by weight of the indicated ingredient in the toothpaste composition.

In some embodiments, the present invention provides a method of treating gingivitis comprising contacting the oral surfaces, e.g., the teeth and gums, of a patient in need thereof with a toothpaste composition containing at least 5% sodium chloride by weight of the toothpaste composition in a toothpaste base comprising calcium carbonate and humectant. In some further embodiments, the toothpaste for use in the method contains from 5% to 15% sodium chloride, for example from 8% to 12% sodium chloride. In some embodiments, the toothpaste contains about 10% sodium chloride.

In some embodiments, the toothpaste for use in treating gingivitis comprises natural calcium carbonate, in an amount of from 10% to 45% by weight of the toothpaste composition, or from 25% to 40%, or from 30% to 35%; or about 32%.

In further embodiments, the toothpaste for use in treating gingivitis further includes one or more humectants. In some embodiments, the humectant is sorbitol, which is present in an amount of from 16% to 26% by weight of the toothpaste composition; or from 18% to 24%; or about 21%.

In some embodiments, the toothpaste for use in treating gingivitis further includes one or more detergents or surfactants. In some embodiments, the toothpaste further includes sodium lauryl sulfate and a poloxamer, for example and without limitation poloxamer 407. In some embodiments, the sodium lauryl sulfate is present in an amount of from 1% to 3% by weight of the toothpaste composition, for example about 2%, and poloxamer 407 is present in an amount of from 0.5% to 2%, for example about 1%.

In some embodiments, the toothpaste for use in treating gingivitis further includes one or more binding agents. In some embodiments, the binding agent includes or consists of a carboxymethylcellulose, for example and without limitation CMC 2000s;, in an amount of from 0.5% to 1.2% by weight of the toothpaste composition; or from 0.7% to 1%; or for example 0.8% to 0.9%.

In some embodiments, the toothpaste for use in treating gingivitis further includes a fluoride source, for example and without limitation monofluorophosphate (MFP), sodium fluoride, or stannous fluoride. In some embodiments, the fluoride source is MFP, which is present in an amount of from 0.5% to 1 % by weight of the toothpaste composition; or 0.6% to 0.9%, for example 0.7% to 0.8%.

In some embodiments, the toothpaste for use in treating gingivitis further includes a thickener, for example and not limitation thickener silica, for example in an amount of from 1% to 3% by weight of the toothpaste composition, for example about 2%.

In some embodiments, the toothpaste for use in treating gingivitis further includes one or more adjuvants selected form sweetening agents, flavoring agents and

coloring agents. In some embodiments, the toothpaste contains flavoring in an amount of from 0.5% to 3.0% by weight of the toothpaste composition; 0.3% to 1.6%; or about 1.2%.

In some embodiments, the toothpaste for use in treating gingivitis includes from 5% to 15% sodium chloride, for example from 8% to 12% sodium chloride, from 30% to 35% calcium carbonate; from 16% to 26% sorbitol; from 1% to 3% SLS, and from 0.5% to 2% poloxamer 407. In some such embodiments, the toothpaste further includes from 0.5% to 1.0% MFP; and from 0.5% to 1.2% CMC 2000s. In some further such embodiments, the toothpaste further includes a thickener, for example thickener silica, for example in an amount of from 1% to 3%, and flavoring, for example in an amount of from 0.5% to 2.0%.

In some embodiments, the toothpaste for use in treating gingivitis includes about 32% calcium carbonate; about 21% sorbitol; about 10% sodium chloride; about 2% thickener silica; about 2% sodium lauryl sulfate; about 1% poloxamer 407;0.8%-0.9% CMC2000s; and 0.7%-0.8% monofluorophosphate.

### DETAILED DESCRIPTION

It has been discovered in accordance with the present invention that high levels of the natural ingredient sodium chloride can function in toothpaste for use in treating gingivitis, as an effective antibacterial agent. The antibacterial activity provides significant benefits by preventing or retarding bacterial growth both in the toothpaste during storage, and in use. In some embodiments, the invention provides toothpaste for use in treating gingivitis having such high levels of sodium chloride formulated with a sorbitol-based humectant system, together with a calcium carbonate abrasive.

The present invention provides toothpastes for use in treating gingivitis that contain at least about 5% sodium chloride, together with abrasive and humectant. In some embodiments, the toothpastes for use in treating gingivitis contain from 5% to 15% sodium chloride, by weight of the toothpaste, for example 8% to 12% sodium chloride. In some embodiments, the toothpaste for use in treating gingivitis contains about 10% sodium chloride. While not wishing to be bound by any theory, the inclusion of high levels of sodium chloride in accordance with the invention is believed to impart antibacterial properties to the compositions, providing benefits in terms of both minimizing bacterial growth during storage and antibacterial efficacy during use.

The toothpastes for use in treating gingivitis further comprise an abrasive, e.g. selected from abrasive silica and/or calcium salts, e.g. calcium carbonate and/or a calcium phosphate abrasive, e.g., tricalcium phosphate (Ca₃(PO₄)₂), hydroxyapatite (Ca_{1 0}(PO₄)((OH)₂), or dicalcium phosphate dihydrate (CaHPO₄2H₂O, also sometimes referred to herein as DiCal) or calcium pyrophosphate. In a particular embodiment, the abrasive includes or is composed of calcium carbonate. Any of the calcium carbonates known to be useful in the dentifrice art are suitable for inclusion in the toothpastes for use in treating gingivitis. In some embodiments, the calcium carbonate is natural calcium carbonate (NCC), preferably in a particle size or distribution of particle sizes wherein 99.5% or greater of the particles passes through a 325 mesh (44 micron). The amount of calcium carbonate in the toothpastes for use in treating gingivitis is for example from 10% to 60%, e .g. 10% to 45%. In some embodiments, the amount of calcium carbonate in the toothpastes for use in treating gingivitis is from 25% to 40% or from 30% to 35%. In some embodiments calcium carbonate is present in an amount of about 32%.

The toothpastes for use in treating gingivitis may also contain a fluoride source—i.e., a fluoride-containing compound having a beneficial effect on the care and hygiene of the oral cavity e.g. diminution of enamel solubility in acid and protection of the teeth against decay. Examples of suitable fluoride sources include sodium fluoride, stannous fluoride, potassium fluoride, potassium stannous fluoride (SNFZ-KF), potassium fluorozirconate, sodium hexafluorostannate, stannous chlorfluoride, and sodium monofluorophosphate (MFP). Where present the fluoride source would provide fluoride ion in amounts sufficient to supply about 25 ppm to about 25,000 ppm of fluoride ions, generally at least about 500 ppm, e .g., about 500 to about 2000 ppm, e.g., about 1000 to about 1600 ppm, e.g., about 1450 ppm. The appropriate level of fluoride will depend on the particular application. A toothpaste for general consumer use would typically have about 1000 to about 1500 ppm, with pediatric toothpaste having somewhat less. A dentifrice or coating for professional application could have as much as about 5,000 or even about 25,000 ppm fluoride. The amount by weight of these materials, which dissociate or release fluoride or fluorine-containing ions, will depend on the molecular weight of the counterion as well as on the particular application, but suitably maybe present in an effective but non-toxic amount, usually within the range of 0.1 to 2% by weight. In some embodiments, a fluoride source selected from sodium fluoride, stannous fluoride, sodium monofluorophosphate and mixtures thereof, is used, for example the toothpaste of the invention may comprise an effective amount of sodium monofluorophosphate. In some embodiments, the fluoride source is sodium monofluorophosphate in an amount of from 0.5% to 1.0% by weight; or 0.6% to 0.9%, for example 0.7% to 0.8%.

The toothpastes for use in treating gingivitis further include humectant, i.e. one or more humectants. Examples of suitable humectants include polyhydric alcohols (polyols) such as propylene glycol, glycerin, sorbitol, xylitol or low molecular weight polyethyleneglycols (PEGs). In various embodiments, humectant scan prevent hardening of paste or gel compositions upon exposure to air, and also function as sweeteners. In some embodiments, the humectant system consists primarily or solely of sorbitol, e.g., in an amount of from 16% to 26%; or from 18% to 24%; or about 21% by weight of the toothpaste composition. However, the presence of other humectants still providing satisfactory toothpaste properties is also contemplated.

The toothpastes for use in treating gingivitis can further include one or more detergents or surfactants. Surfactants useful for the present invention include, without limitation: anionic, nonionic, and amphoteric surfactants. Surfactants maybe used, for example, to provide enhanced stability of the formulation, to help in cleaning the oral cavity surfaces through detergency, and to increase foaming of the composition upon agitation, e.g., during brushing. Suitable anionic surfactants include, for example, water-soluble salts of C₈₋₂₀ alkyl sulfates, sulfonated monoglycerides of C₈₋₂₀ fatty acids, sarcosinates and taurates; for example sodium lauryl sulfate, sodium coconut monoglyceride sulfonate, sodium lauryl sarcosinate, sodium lauryl isoethionate, sodium laureth carboxylate and sodium dodecyibenzenesulfonate, and mixtures thereof. Suitable nonionic surfactants include, for example, poloxamers, polyoxyethylene sorbitan esters, fatty alcohol ethoxylates, alkylphenol ethoxylates, tertiary amine oxides, tertiary phosphine oxides, dialkyl sulfoxides, and mixtures thereof. In one embodiment, the toothpaste comprises sodium lauryl sulfate, for example in an amount of from 1% to 3%, or about 2%. The toothpaste may also or alternatively contain one or more nonpolar surfactans, for example polymers and co-polymers of ethylene glycol and propylene glycol, e.g., poloxamers, i.e., nonionic triblock copolymers composed of a central hydrophobic chain of polyoxypropylene (polypropylene oxide)) flanked by two hydrophilic chains of polyoxyethylene (polyethylene oxide)). The approximate lengths of the two PEG blocks is, in some embodiments, an average of about 50-150 repeat units, e.g., about 100 repeat units while the approximate length of the propylene gycol block is an average of about 25-75 repeat unties, e.g., about 50-60 repeat units. In one embodiment, the poloxamer is poloxamer 407, also known by the BASF trade name Pluronic F127, e.g., in an amount of from 0.5% to 2%, for example about 1%. For example, in certain embodiments, the toothpastes for use in treating gingivitis may contain both sodium lauryl sulfate and a poloxamer such as poloxamer 407.

In some embodiments, the toothpastes for use in treating gingivitis further include one or more binding and/or thickening agents. Binding agents may include polymers include polyethylene glycols, polysaccharides (e.g., cellulose derivatives, for example carboxymethyl cellulose, or polysaccharide gums, for example xanthan gum or carrageenan gum). Acidic polymers, for example polyacrylate gels, maybe provided in the form of their free acids or partially or fully neutralised water soluble alkali metal (e.g., potassium and sodium) or ammonium salts; and include synthetic anionic polymeric polycarboxylates, such as 1:4 to 4:1 copolymers of maleic anhydride or acid with another polymerizable ethylenically unsaturated monomer, preferably methyl vinyl ether/maleic anhydride having a molecular weight (MW.) of about 30,000 to about 1,000,000, most preferably about 300,000 to about 300,000. In some embodiments, the binding agent is derived from cellulose. In some embodiments, the binding agent includes or consists of cellulose ether, for example carboxymethylcellulose, for example CMC 2000s, in an amount of from 0.5% to 1.2%; or from 0.7% to 1.0%; or 0.8% to 0.9%.

In some embodiments, the toothpastes for use in treating gingivitis further include one or more thickeners (i.e., thickening agents), which aid in obtaining the proper viscosity of the composition. Generally, the thickener is present in the composition in an amount of from 1% to 5%. Examples of thickening agents include, without limitation, the binding agents described above, which also modify viscosity, for example carboxyvinyl polymers, carrageenan (also known as Irish moss and more particularly iota-carrageenan), cellulosic polymers such as hydroxyethylcellulose, carboxymethylcellulose (carmellose) and salts thereof (e.g., carmellose sodium), natural gums such as karaya, xanthan, gum arabic and tragacanth, colloidal magnesium aluminum silicate, colloidal silica, and mixtures thereof. One thickener is thickener silica, for example in an amount of from 1% to 3%.

As will be evident to one of skill in the art, some components of the invention may perform multiple functions, and the identification of a compound as having one function herein is not meant to exclude its use for other functions in a particular composition. For example, a compound such as carboxymethylcellulose may act as a binder, but also has humectant and thickening properties, or a compound such as a poloxamer, while identified above as a nonionic surfactant, also has humectant and thickening properties.

It is also understood that compounds in formulation may naturally react, disassociate, and/or form complexes with one another. Accordingly, certain ingredients maybe formed in situ (for example, it is understood that sodium chloride maybe formed by reacting sodium hydroxide with hydrochloric acid), and also may in formulation exist in different forms (for example, to the extent the sodium chloride is dissolved, it will naturally disassociate into separate sodium and chloride ions, as opposed to a solid salt). As is usual in the art, the compositions of the invention are described in terms of exemplary formulation ingredients, without intending to exclude combinations of other ingredients that result in the same final compositions, or to exclude the natural reaction products of the described ingredient combinations.

In some embodiments, the toothpaste includes from 5% to 15% sodium chloride, for example from 3% to 12% sodium chloride; from 30% to 35% calcium carbonate; from 16% to 26% sorbitol; from 1% to 3% SLS, and from 0.5% to 2% poloxamer 407. In some such embodiments, the toothpaste further includes from 0.5% to 1.0% MFP; and from 0.5% to 1.2% CMC 2000s. In some further such embodiments, the toothpaste further includes thickener silica, for example in an amount of from 1% to 3%.

In one embodiment, the toothpaste composition includes about 32% calcium carbonate, about 21% sorbitol, about 10% sodium chloride, about 2% thickener silica, about 2% SLS, about 1.0% poloxamer 407, 0.8% to 0.9% CMC 2000s, aid 0.7% to 0.8% MFP.

In some embodiments described above, the toothpastes for use in treating gingivitis can further include one or more sweetening agents, flavoring agents and coloring agents. Any suitable flavoring or sweetening material maybe employed. Examples of suitable flavoring constituents include flavoring oils, e.g. oil of spearmint, peppermint, wintergreen, clove, sage, eucalyptus, marjoram, cinnamon, lemon, and orange, and methyl salicylate. Suitable sweetening agents include sucrose, lactose, maltose, xylitol, sodium cyclamate, perillartine, AMP (aspartyl phenyl alanine methyl ester), saccharine and the like. Suitably, flavor and sweetening agents may each or together comprise from about 0.1% to 5% more of the oral care composition. In some embodiments, the toothpastes for use in treating gingivitis include one or more flavoring agents in an amount of from about 0.5% to about 3.0%; about 0.8% to about 1.6%; or about 1.2%.

Various other materials maybe incorporated in the oral preparations of this invention such as whitening agents including urea peroxide, calcium peroxide, titanium dioxide, hydrogen peroxide, complexes of polyvinylpyrolidone (PVP) and hydrogen peroxide, preservatives, vitamins such as vitamin B6, B12, E and K, silicones, chlorophyll compounds, potassium salts for the treatment of dental hypersensitivity such as potassium nitrate as well as antitartar agents such as sodium tripolyphosphate and di- and tetra-alkali metal pyrophosphate salts such as di- and tetrasodium pyrophosphate. These agents, when present, are incorporated in the compositions of the present invention in amounts which do not substantially adversely affect the properties and characteristics desired.

In general, each of the foregoing adjuvants maybe typically incorporated in the instant toothpastes in amounts up to 5% provided they do not adversely affect the stability and cleaning properties of the non-bleeding striped dentifrice of present invention.

The invention thus provides, in one embodiment, a toothpaste for use in treating gingivitis in a patient in need thereof, the toothpaste comprising at least 5% sodium chloride; from 5% to 15% sodium chloride; from 8% to 12% sodium chloride; or about 10% sodium chloride (Composition 1), in a toothpaste base comprising calcium carbonate abrasive and humectant, for example

1.1.Composition 1, wherein the abrasive comprises natural calcium carbonate, in an amount of from 10% to 45%; 25% to 40%; 30% to 35%; or about 32%.
1.2. Any foregoing composition wherein the humectant comprises a polyol, e.g., sorbitol, e.g., sorbitol in an amount of from 16% to 26%, or from 18% to 24%; or about 21%.
1.3. Any foregoing composition further comprising one or more anionic detergents or surfactants, e.g., sodium lauryl sulfate, in an amount of from 1% to 3%, or about 2%; and one or more nonionic surfactants, e .g., a poloxamer, e.g., poloxamer 407, in an amount of from 0.5% to 2%; or about 1%.
1.4. Any foregoing composition wherein the binder comprises a cellulose derivative, e.g., carboxymethylcellulose (CMC), e.g. having a medium to high degree of polymerization, e.g. 1000 to 3000, for example about 2000, e.g., in sodium salt form, e.g., CMC 2000s, in an amount effective to provide the desired viscosity, e.g., from 0.5% to 1.2%; from 0.7% to 1.0%; or 0.8% to 0.9%.
1.5. Any foregoing composition further comprising an effective amount of a fluoride ion source; e.g., sodium monofluorophosphate (MFP), in an amount of from 0.5% to 1.0%; or 0.7% to 0.8%, e.g., about 0.76%.
1.6. Any foregoing composition comprising:
from 30% to 35% calcium carbonate;
from 16% to 26% sorbitol;
from 1% to 3% sodium lauryl sulfate;
from 0.5% to 2% poloxamer 407;
from 0.5% to 1.0% sodium monofluorophosphate;
from 0.5% to 1.2% CMC; and
thickener silica in an amount of from 1% to 3%; or about 2%.
   1.7. The toothpaste of any the foregoing compositions, further comprising one or more adjuvants selected from sweetening agents flavoring agents and coloring agents, e.g., comprising a flavoring agent in an amount of from 0.5% to 3.0%; 0.8% to 1.6%; or about 1.2%.
   1.8. Any foregoing composition comprising about 32% calcium carbonate, about 21% sorbitol, about 10% sodium chloride, about 2% thickener silica, about 2% SLS, about 1.0% poloxamer 407, 0.8% to 0.9% CMC, and 0.7% to 0.8% MFP.

The invention further provides, in another embodiment, a method (Method 1) for treating gingivitis in a patient in need thereof, comprising applying a toothpaste to the gums of the patient wherein the toothpaste comprises at least 5% sodium chloride; from 5% to 15% sodium chloride; from 8% to 12% sodium chloride; or about 10% sodium chloride, in a toothpaste base comprising calcium carbonate abrasive and humectant comprising a calcium carbonate abrasive, e.g., a toothpaste of any of Compositions 1, et seq. For example the invention provides:

1.1. Method 1, where in the abrasive comprise s natural calcium carbonate, in an amount of from 10% to 45%; 25% to 40%; 30% to 35%; or about 32%.
1.2. Any foregoing method wherein the humectant comprises a polyol, e.g., sorbitol, e.g., sorbitol in an amount of from 16% to 26%, or from 18% to 24%; or about 21%.
1.3. Any foregoing method where in the toothpaste further comprises one or more anionic detergents or surfactants, e.g., sodium lauryl sulfate, in an amount of from 1% to 3%, or about 2%; and one or more nonionic surfactants, e.g., a poloxamer, e.g., poloxamer 407, in an amount of from 0.5% to 2%; or about 1%.
1.4. Any foregoing method where in the binder comprises a cellulose derivative, e.g., carboxymethylcellulose (CMC), e.g. having a medium to high degree of polymerisation, e.g. 1000 to 3000, for example about 2000, e.g., in sodium salt form, e.g., CMC 2000s, in an amount effective to provide the desired viscosity, e.g., from 0.5% to 1.2%; from 0.7% to 1.0%; or 0.8% to 0.9%.
1.5. Any foregoing method wherein the toothpaste further comprises an effective amount of a fluoride ion source; e.g., sodium monofluorophosphate (MFP), in an amount of from 0.5% to 1.0%; or 0.7% to 0.8%, e .g, about 0.76%.
1.6. Any foregoing method wherein the toothpaste comprises: from 30% to 35% calcium carbonate;
from 16% to 26% sorbitol;
from 1% to 3% sodium lauryl sulfate;
from 0.5% to 2% poloxamer 407;
from 0.5% to 1.0% sodium monofluorophosphate;
from 0.5% to 1.2% CMC; and
thickener silica in an amount of from 1% to 3%; or about 2%.
   1.7. Any preceding method, wherein the toothpaste further comprises one or more adjuvants selected from sweetening agents flavoring agents and coloring agents, e.g., comprising a flavoring agent in an amount of from 0.5% to 3.0%; 0.8% to 1.6%; or about 1.2%.
   1.8. Any foregoing method wherein the toothpaste comprises about 32% calcium carbonate, about 21% sorbitol, about 10% sodium chloride, about 2% thickener silica, about 2% SLS, about 1.0% poloxamer 407, 0.8% to 0.9% CMC, and 0.7% to 0.8% MFP.
   1.9. Any foregoing method where in the toothpaste is applied at least daily, e.g., once, twice or thrice daily, until an effect is seen, e.g., over a period of at least a week, e.g., at least two weeks, e.g., at least a month.

The invention further provides, in another embodiment, the use of sodium chloride in the manufacture of a toothpaste for treating gingivitis in a patient in need thereof, e.g., a toothpaste according to any of Compositions 1, et seq., in a method according to any of Methods 1, et seq.

The following examples are further illustrative of the nature of the present invention, but it is understood that the invention is not limited thereto. All amounts and proportions referred to herein and in the appended claims are by weight, unless otherwise indicated.

### Example 1—Micro Robustness Test

The micro robustness test (MRT) is a quantitative measure of a composition's ability to withstand microbial challenge. Thus, the result is an assessment of the antimicrobial efficacy of a composition against a pool of microorganisms.

The following eleven microorganisms are included in a microorganism pool: Burkholderia cepacia, Enterobacter cloacae, Escherichia coli, Klesiella oxytoca, Klebsiella pneumoniae, Serratia marcescens, Povidencia rettgeri, Pseudomonas aeruginosa, Pseudomonas putida, Staphylococcus aureus, and Staphylococcus saprophyticus.

The total microorganism's solution level is 10⁷ cfu/ml. Samples are challenged three times at 60 minute internals with 10⁷ bacteria from the microorganisms pool described above. After 4, 6 and 24 hours, aliquots are tested to measure the log reduction of bacterial level. Table 1 below shows the results for the antimicrobial test on a toothpaste of the invention having the following composition:

| | |
|---|---|
| Calcium Carbonate | 32% |
| Water | 27.9347% |
| Sorbitol | 21% |
| NaCl | 10% |
| Thickener silica | 2% |
| SLS | 2% |
| Flavor | 1.2% |
| Poloxamer 407 | 1.0% |
| CMC 2000s | 0.85% |
| MFP | 0.76% |
| and minor ingredients. | |

TABLE 1
Results for Antimicrobial Test

| Initial added bacteria | 4 hours | 6 hours | 24 hours |
|---|---|---|---|
| level | inoculum | inoculum | inoculum |
| 6.4 × 10⁷ cfu/ml | <10 cfu/ml | NG (no | NG (no growth) |
| | | growth) | |

Table 1 shows that the bacteria tested is shown to be effectively decreased to less than <10 cfu/ml from the initial level of 6.4×10⁷cfu/ml in 4 hours, with no growth in 6 hours or 24 hours inoculum. Thus, the high salt level toothpaste of the invention has antibacterial ability and can inhibit the growth of varieties bacteria in the toothpaste.

### Example 2—Biofilm Reduction Test

Methods: Dental plaque is collected from 4 healthy volunteers and pooled together as inoculum. The O.D of the inoculum is matched to 0.3 absorbance at 610 nm. Sterile HAP disks are incubated under anaerobic conditions at 37° C. for 24 hours with 1ml of sterile artificial saliva (with 0.01% sucrose) and 1 ml of pooled saliva in a 24 well micro plate. Freshly pre pared treatment solution (1 part toothpaste of Example 1 above to 2 parts sterile distilled water) is added to the well and allowed to contact with the HAP disk for 10 minutes.

The liquid phase is removed and replaced by 2 ml of sterile artificial saliva. The disks are treated in triplicates for each control and test dentifrice for 8 days. At intervals of 2, 4 and 8 days the discs are collected aseptically and transferred into half strength pre-reduced thioglycollate medium. 100 ml of the dilution 10-4, 10-5 and 10-6are plated in duplicates for each disk on Neomycin Vancomycin (NV) Agar, for Total Gram negative Anaerobes. Plates are surface spread using a sterile spreader and incubated anaerobically at 37° C. for 72 hours be fore counting the colonies. The pH is monitored for the entire period of the study using the liquid phase. The compositions of the samples used in the test are shown in Tables 2a and 2b below. Samples 1-5 contain the same formula backbone with silica base and 1.0 % ZnO and 0.5 % Zinc Citrate in place of sodium chloride. These samples also contain different levels essential oils: Sample 1: Silica base formula with 1.0% ZnO and 0.5% Zinc Citrate with full essential oil cocktail level.
Sample 2: Silica base formula with 1.0% ZnO and0.5% Zinc Citrate with half essential oil cocktail level.
Sample 3: Silica base formula with 1.0% ZnO and 0.5% Zinc Citrate with full essential oil c ocktail level but half level of Thymol.
Sample 4: Silica base formula with 1.0% ZnO and 0.5% Zinc Citrate with full essential oil but half level of Thymol and Eucalyptus.
Sample 5: Silica base formula with 1.0% ZnO and 0.5% Zinc Citrate, no essential oil.
Sample 6: Placebo, Silica base formula without ZnO, Zinc Citrate and essential oil.
Sample 7: NCC base formula with 10% NaCl (formulation of Example 1)
Sample 8: Commercial product with 0.58% Zinc Citrate

TABLE 2a
Compositions of Samples for Biofilm Reduction Test

| Sample | 1 | 2 | 3 | 4 |
|---|---|---|---|---|
| RAW MATERIAL | % | % | % | % |
| CP water | 13.53 | 19.09 | 18.65 | 18.99 |
| sorbitol-70% solution | 55.00 | 50.00 | 50.00 | 50.00 |
| Polyethylene Glycol 600 | 2.00 | | | |
| Glycerine | | 2.00 | 2.00 | 2.00 |
| sodium saccharin | 0.30 | 0.30 | 0.30 | 0.30 |
| ZnO | 1.00 | 1.00 | 1.00 | 1.00 |
| Zinc Citrate | 0.50 | 0.50 | 0.50 | 0.50 |
| TS PP | 0.50 | 0.50 | 0.50 | 0.50 |
| CMC-Type 12 (2000S) | 0.80 | 0.80 | 0.80 | 0.80 |
| Xanthan | 0.30 | 0.30 | 0.30 | 0.30 |
| MFP | 1.10 | 1.10 | 1.10 | 1.10 |
| Silica abrasive Zeo 114 | 10.00 | 10.00 | 10.00 | 10.00 |
| AC 43 | 5.00 | 5.00 | 5.00 | 5.00 |
| Silica thickener DT 267 | 4.00 | 4.00 | 4.00 | 4.00 |
| SLS powder | 2.00 | 2.00 | 2.00 | 2.00 |
| Betaine | 1.25 | 1.25 | 1.25 | 1.25 |
| Clove Oil | 0.177 | 0.0885 | 0.177 | 0.177 |
| Thymol | 0.25 | 0.125 | 0.125 | 0.125 |
| Eucalyptus Oil | 0.68 | 0.34 | 0.68 | 0.34 |
| Lemon Oil | 0.01 | 0.005 | 0.01 | 0.01 |
| Basil Oil (Firmerich) | 0.005 | 0.0025 | 0.005 | 0.005 |
| Maxfresh Cool Mint flavor | 1.10 | 1.10 | 1.10 | 1.10 |
| TiO2 | 0.50 | 0.50 | 0.50 | 0.50 |
| CMC-Type 8 | | | | |
| poloxomer 407 USP | | | | |
| Refined Soda | | | | |
| Natural Calcium Carbonate | | | | |
| Mint Flavor for Herbal Salt | | | | |
| Sodium Chloride | | | | |
| Sorbosil BFG51-Blue | | | | |
| Tocopheryl Acetate | | | | |
| Sodium Ascorbyl Phosphate | | | | |
| Sodium Bicarbonate | | | | |
| CI Food Blue 5 | | | | |
| TOTAL | 100.00 | 100.00 | 100.00 | 100.00 |

TABLE 2b
Compositions of Samples for Biofilm Reduction Test (cont.)

| Sample | 5 | 6 | 7 | 8 |
|---|---|---|---|---|
| RAW MATERIAL | % | % | % | % |
| CP water | 19.65 | 21.15 | 27.9347 | |
| sorbitol-70% solution | 50.00 | 50.00 | 21.00 | 37.00 |
| Polyethylene Glycol 600 | | | | |
| Glycerine | 2.00 | 2.00 | | |
| sodium saccharin | 0.30 | 0.30 | 0.30 | |
| ZnO | 1.00 | | | |
| Zinc Citrate | 0.50 | | | 0.58 |
| TS PP | 0.50 | 0.50 | | |
| CMC-Type 12 (2000S) | 0.80 | 0.80 | | |
| Xanthan | 0.30 | 0.30 | 0.20 | |
| MFP | 1.10 | 1.10 | 0.76 | |
| Silica abrasive Zeo 114 | 10.00 | 10.00 | | 15.00 |
| AC 43 | 5.00 | 5.00 | | |
| Silica thickener DT 267 | 4.00 | 4.00 | 2.00 | |
| SLS powder | 2.00 | 2.00 | 2.00 | 2.00 |
| Betaine | 1.25 | 1.25 | | |
| Clove Oil | | | | Commercial |
| Thymol | | | | Product |
| Eucalyptus Oil | | | | |
| Lemon Oil | | | | |
| Basil Oil (Firmerich) | | | | |
| Maxfresh Cool Mint flavor | 1.10 | 1.10 | | |
| TiO2 | 0.50 | 0.50 | | |
| CMC-Type 8 | | | 0.85 | |
| poloxomer 407 USP | | | 1.00 | |
| Refined Soda | | | 0.40 | |
| Natural Calcium Carbonate | | | 32.00 | |
| Mint Flavor for Herbal Salt | | | 1.20 | |
| Sodium Chloride | | | 10.00 | |
| Sorbosil BFG51-Blue | | | 0.20 | |
| Tocopheryl Acetate | | | 0.05 | |
| Sodium Ascorbyl Phosphate | | | 0.01 | |
| Sodium Bicarbonate | | | 0.10 | |
| CI Food Blue 5 | | | 0.00 | |
| TOTAL | 100.00 | 100.00 | 100.00 | |

TABLE 3
Results of Biofilm Reduction Test

| | Total Gram |
|---|---|
| | Negative Anaerobes |
| | on NV Agar, Log |
| Sample | CFU/ml |
| Sample 1: Silica base with 1.0% ZnO and 0.5% Zinc | 4.59 |
| Citrate with full essential oil cocktail level | |
| Sample 2: Silica base with 1.0% ZnO and 0.5% Zinc | 4.74 |
| Citrate with half essential oil cocktail level | |
| Sample 3: Silica base with 1.0% ZnO and 0.5% Zinc | 4.79 |
| Citrate with full essential oil cocktail level but half | |
| level of Thymol | |
| Sample 4: Silica base with 1.0% ZnO arid 0.5% Zinc | 4.82 |
| Citrate with full essential oil but half level of | |
| Thymol and Eucalyptus | |
| Sample 5: Silica base with 1.0% ZnO and 0.5% Zinc | 4.86 |
| Citrate, no essential oil | |
| Sample 6: Placebo, Silica base formula without ZnO, | 6.09 |
| Zinc Citrate and essential oil | |
| Sample 7: NCC base formula with 10% NaCl | 4.47 |
| Sample 8: Commercial product with 0.58% Zinc | 5.02 |
| Citrate | |

It can be seen from the data in Table 3 that the efficacy of the actives are found to be in the following order (from the most efficacious to less):
a. Sample 7: NCC base formula with 10% NaCl
b. Sample 1: Silica base formula with 1.0% ZnO and 0.5% Zinc Citrate with full essential oil cocktail level
c. Sample 2: Silica base formula with 1.0% ZnO and 0.5% Zinc Citrate with half essential oil cocktail level
d. Sample 3: Silica base formula with 1.0% ZnO and 0.5% Zinc Citrate with full essential oil cocktail level but half level of Thymol
e. Sample 4: Silica base formula with 1.0% ZnO and0.5% Zinc Citrate with full essential oil but half level of Thymol and Eucalyptus.
f. Sample 5: Silica base formula with 1.0% ZnO and 0.5% Zinc Citrate, no essential oil
g. Sample 8: Commercial product with 0.58% Zinc Citrate
h. Sample 6: Placebo, Silica base formula without ZnO, Zinc Citrate and essential oil

The results indicate that the toothpaste having 10% sodium chloride in combination with a natural calcium carbonate base, as opposed to a silica abrasive base, has better efficacy on biofilm reduction than a variety of silica-based toothpastes comprising a variety of antibacterial agents. This reduction in biofilm corresponds to a reduction in the amount of dental plaque when the toothpaste is used regularly, thereby providing a reduction in the incidence and severity of gingivitis.

While the present invention has been described with reference to embodiments, it will be understood by those skilled in the art that various modifications and variations maybe made therein without departing from the scope of the present iiwention as defined by the appended claims.

1. A method of treating gingivitis, comprising applying a toothpaste comprising at least 5% sodium chloride by weight of the toothpaste, in a toothpaste base comprising a non-silica calcium carbonate abrasive and humectant, to the oral surfaces of a patient in need thereof.

2. The method of claim 1, wherein the abrasive comprises natural calcium carbonate in an amount of from 10% to 45% by weight of the toothpaste.

3. The method of claim 1 wherein the toothpaste comprises 8% to 12% sodium chloride by weight of the toothpaste.

4. The method of claim 1 wherein the humectant comprises one or more polyols in an amount of from 16% to 26% by weight of the toothpaste.

5. The method of claim 1 wherein the toothpaste further comprises one or more detergents or surfactants in an amount of from 1% to 3%; and a poloxamer in an amount of from 0.5% to 2% by weight of the toothpaste.

6. The method of claim 1 wherein the toothpaste further comprises one or more binding agents, in an amount of from 0.5% to 1.2% by weight of the toothpaste.

7. The method of claim 1 wherein the toothpaste further comprises a fluoride source; e.g., monofluorophosphate, in an amount of from 0.5% to 1.0% by weight of the toothpaste.

8. The method of claim 1 wherein the toothpaste comprises:
from 8% to 12% sodium chloride;
from 30% to 35% calcium carbonate;
from 16% to 26% sorbitol,
from 1% to 3% sodium lauryl sulfate, and
from 0.5% to 2% poloxamer 407.

9. The method of claim 1 wherein, the toothpaste further comprises:
from 0.5% to 1.0% sodium monofluorophosphate (MFP); and
from 0.5% to 1.2% sodium carboxymethylcelulose (CMC).

10. The method of claim 1 wherein the toothpaste further comprises thickener silica in an amount of from 1% to 3%.

11. The method of claim 1 wherein the toothpaste further comprises one or more adjuvants selected from sweetening agents, flavoring agents and coloring agents.

12. The method of claim 1 wherein live toothpaste further comprises a flavoring agent in an amount of from 0.5% to 3.0%.

13. The method of claim 1 wherein the toothpaste comprises about 32% calcium carbonate, about 21% sorbitol, about 10% sodium chloride, about 2% thickener silica, about 2% SLS, about 1.0% poloxamer 407, 0.8%) to 0.9% carboxymethyl cellulose, and 0.7% to 0.8% sodium monofluorophosphate.

14. The method of claim 1, wherein the toothpaste is applied at least, daily for at least two weeks.

15. A toothpaste comprising at least 5% sodium chloride by weight of the toothpaste, in a toothpaste base comprising a non-silica calcium carbonate abrasive and humectant, for use in the treatment of gingivitis, e.g., in a method of claim 1.

16. (canceled)
